(19) United States
(12) Patent Application Publication
(10) Pub. No.: US 2021/0299402 A1
(43) Pub. Date: Sep. 30, 2021

(54) CATHETER TUBE AND METHOD FOR MANUFACTURING THE SAME

(71) Applicant: HIRAKAWA HEWTECH CORPORATION, Tokyo (JP)

(72) Inventor: Sho SAKAMOTO, Koga-shi (JP)

(21) Appl. No.: 17/216,124

(22) Filed: Mar. 29, 2021

(30) Foreign Application Priority Data
Mar. 31, 2020 (JP) 2020-064848

(51) Int. Cl. A61M 25/00

(52) U.S. Cl. CPC A61M 25/0053; A61M 25/0045

(57) ABSTRACT

A catheter tube includes an inner layer tube as an axial center portion, a reinforcing wire member composed of a first helical portion helically wound around the inner layer tube from a base portion of the inner layer tube to an intermediate position near a tip portion, and a second helical portion helically wound around by being folded in a direction intersecting with the first helical portion at the intermediate position, and an outer layer tube covering the inner layer tube and the reinforcing wire member.

### CROSS-REFERENCE TO RELATED APPLICATION

The present application is based on Japanese Patent Application No. 2020-64848 filed on Mar. 31, 2020, the entire contents of which are incorporated herein by reference.

### BACKGROUND OF THE INVENTION

### 1. Field of the Invention

The present invention relates to a catheter tube and a method for manufacturing the same, more particularly, to the catheter tube with improved safety and the method for manufacturing the same.

### 2. Description of the Related Art

The catheter tube is a lengthy tube having a tip-side that is a side inserted into a body (blood vessels, etc.) and a hand-side for performing handling, operation or the like. Such a catheter tube has a laminated structure in which an inner layer tube, a reinforcing layer provided on the inner layer tube, and an outer layer tube are integrated.

In such a catheter tube, at a tip-side end of the reinforcing layer, a tip-side terminal of a reinforcing wire member is provided as a part of the reinforcing layer. Thus, a very dangerous failure may occur in which the tip-side terminal of the reinforcing wire member is exposed or protruding to the surface of the catheter tube at the time of coating the outer layer tube or connecting a flexible tube thereto. Further, there is a possibility that the tip-side terminal of the reinforcing wire member protrudes to the surface of the catheter tube due to excessive flexion during use.

Therefore, the inspection of the tip-side terminal of the reinforcing wire member is strictly performed at the time of manufacturing, and a catheter tube in which the tip-side terminal of the reinforcing wire member does not expose or protrude therefrom is provided. For the same reason, it is also difficult to use a reinforcing wire member with excellent repulsive force, which is naturally expected to provide a high reinforcing effect.

As an example of catheter tubes, a catheter tube having a non-reinforcing portion without a reinforcing layer, from which a tip portion of the reinforcing layer provided on the inner layer tube is removed by chemical removal method or the like, has been proposed (see, e.g., JP2001-79095A, JP2001-178814A, JP2001-190679A, JP2000-51363A, and JP2000-51364A).

As the other examples of catheter tubes, a catheter tube comprising a braid with two layers of a large pitch layer and a fine pitch layer, and a catheter in which a synthetic resin fiber is helically wound in a coil shape at a large pitch and a metal wire braid at a fine pitch is provided thereon have been proposed (see, e.g., JP2006-158878A and JP2006-218085A).

As still another example of catheter tubes, a catheter tube having a non-reinforcing portion without a reinforcing layer, from which a terminal portion of a reinforcing member is narrowed by chemical polishing or the like or the terminal portion of the reinforcing member is fixed by a tape or the like, has been proposed.

### PRIOR ART LITERATURE

### Patent Documents

Patent Document 1: JP2001-79095A
Patent Document 2: JP2001-178814A
Patent Document 3: JP2001-190679A
Patent Document 4: JP2000-51363A
Patent Document 5: JP2000-51364A
Patent Document 6: JP2006-158878A
Patent Document 7: JP2006-218085A

### SUMMARY OF THE INVENTION

According to the catheter tubes described in JP2001-79095A, JP2001-178814A, JP2001-190679A, JP2000-51363A, and JP2000-51364A, a step of forming the reinforcing layer on the inner layer tube requires a step of removing the tip portion of the reinforcing layer by chemical removal treatment or the like after forming the reinforcing layer. Therefore, in the structure of the catheter tube described above, it becomes difficult to suppress the manufacturing time and manufacturing cost. Further, when the chemical removal treatment or the like is not sufficient, the yield will not be stable.

Even in the catheter tubes described in JP2000-51364A and JP2006-158878A, since there is a tip-side terminal of the reinforcing wire member as the reinforcing layer, a step of heat treatment of the tip portion of the reinforcing layer is required to form a rounded or tapered shape, so that it is difficult to suppress the manufacturing time and manufacturing cost similarly to the catheters described in JP2001-79095A, JP2001-178814A, JP2001-190679A, JP2000-51363A, and JP2000-51364A, and the yield is not stable.

Accordingly, it is an object of the present invention to provide a catheter tube having a safe structure without a tip-side terminal of a reinforcing wire member at a tip of the catheter tube and a method for manufacturing the same.

For achieving the above object, an aspect of the present invention provides a catheter tube, comprising:

an inner layer tube as an axial center portion;

a reinforcing wire member comprising a first helical portion helically wound around the inner layer tube from a base portion of the inner layer tube to an intermediate position near a tip portion, and a second helical portion helically wound around by being folded in a direction intersecting with the first helical portion at the intermediate position; and

an outer layer tube covering the inner layer tube and the reinforcing wire member.

In the catheter tube, the reinforcing wire member may comprise one or two or more wires wound at a predetermined pitch.

Another aspect of the present invention provides a method for manufacturing a catheter tube including an inner layer tube as an axial center portion, a reinforcing wire member wrapped around the inner layer tube along an axial outer circumference surface of the inner layer tube, and an outer layer tube covering the inner layer tube and the reinforcing wire member,

the method comprising:

setting the inner layer tube;

helically winding the reinforcing wire member around the inner layer tube from a base portion of the inner layer tube to an intermediate position near the tip portion of the inner layer tube and turning back in a direction intersecting the reinforcing wire member from the intermediate position while rotating the inner layer tube and the reinforcing wire member relatively to each other; and

covering the inner layer tube and the reinforcing wire member with the outer layer tube.

Here, the feature “while rotating the inner layer tube and the reinforcing wire member relatively to each other” includes stopping one of the inner layer tube and the reinforcing wire member and rotating the other, and rotating the inner layer tube and the reinforcing wire member in the opposite directions to each other, or the like. The rotation of the inner layer tube is to rotate the inner layer tube around the axial center, and the rotation of the reinforcing wire member is to rotate the reinforcing wire member around the axial center of the inner layer tube. The rotation of both the inner layer tube and the reinforcing wire member includes the rotation of the inner layer tube and the reinforcing wire member at a speed difference from each other.

The method for manufacturing the catheter tube according to the present invention may include repeating the winding of the reinforcing wire member at a position shifted by a predetermined distance in a tip direction from the intermediate position of the inner layer tube after the winding of the reinforcing wire member.

### Effect of Invention

According to the present invention, since there is no tip-side terminal of the reinforcing wire member at the tip of the catheter tube, a safe structure can be obtained, the manufacturing time and manufacturing cost can be suppressed, and the yield can be increased.

### BRIEF DESCRIPTION OF THE DRAWINGS

Next, the present invention will be explained in more detail in conjunction with appended drawings, wherein:

FIGS. 1A and 1B are diagrams showing an example of a catheter tube according to the embodiment of the present invention, in which FIG. 1A is a plan schematic view showing a part of the catheter tube through perspective, and FIG. 1B is a transversal cross-sectional schematic view;

FIG. 2 is a photographic image for showing a catheter tube according to the embodiment (located in the upper stage), a catheter tube having a conventional mesh winding reinforcing portion (located at the lower stage), and a catheter tube having a conventional reinforcing portion in which the tip-side is the end of the coil winding (located in the intermediate stage) in comparison;

FIGS. 3A to 3C are explanatory diagrams for showing an example of a method for manufacturing a catheter tube according to the embodiment;

FIG. 4 is an explanatory diagram for showing another example of a method for manufacturing a catheter tube according to the embodiment;

FIG. 5 is an explanatory diagram showing a working example of a catheter tube according to the embodiment of the present invention, and is a plan schematic view showing a part of the catheter tube through perspective;

FIGS. 6A and 6B are schematic diagrams for showing an example of a winding process of a wire member that reinforces the catheter tube in the working example;

FIG. 7 is a plan schematic view showing a part of a catheter tube in a comparative example through perspective;

FIGS. 8A and 8B are schematic diagrams for showing an example of a winding process of a wire member that reinforces the catheter tube in the comparative example according to the embodiment;

FIG. 9 is a table showing the relationship between the catheter tube according to the example and the catheter tube according to the comparative example and the evaluation result;

FIG. 10A is a schematic diagram for explaining the kink resistance test of the catheter tube;

FIG. 10 B is a graph showing the result of the kink resistance test; and

FIGS. 11A and 11B are graphs showing the result of the breaking strength and tensile modulus test of the catheter tube.

### DETAILED DESCRIPTION OF THE PREFERRED EMBODIMENTS

Next, a catheter tube and a method for manufacturing the catheter tube in the embodiment according to the present invention will be described below with reference to the appended drawings.

(Catheter Tube Configuration)

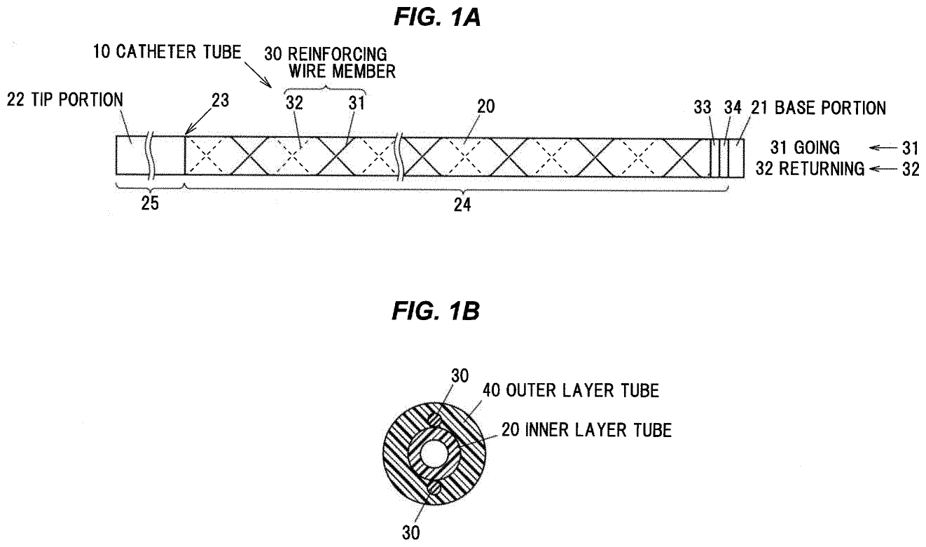

In FIG. 1A, a catheter tube 10 according to the present embodiment is applicable to various medical tubes such as guiding catheters, microcatheters, angiography catheters, endoscopes, and the like. The dimensions of the catheter tube 10 are appropriately set according to the type, treatment site, and the like.

As shown in FIGS. 1A and 1B, the catheter tube 10 includes an inner layer tube 20, a reinforcing wire member 30 wrapped around the inner layer tube 20 by cross winding in a predetermined length range along an axial outer circumference surface of the inner layer tube 20, and an outer layer tube 40 covering the inner layer tube 20 and the reinforcing wire member 30, thereby providing a catheter body portion configured to be inserted into an organism lumen. The cross winding has a shape in which the wire members are folded back continuously in such a manner that winding directions of the wire members are in the opposite direction to each other so that the wire members are wrapped in a double helix.

The catheter tube 10 further includes a reinforcing portion 24 around which the reinforcing wire member 30 is wrapped in cross winding in a range from a base portion 21 on a hand-side (i.e., a proximal side) of the inner layer tube 20 to an intermediate position 23 near a tip portion 22 that is an insertion-side into the body (i.e., a distal side), and a non-reinforcing portion 25 around which the reinforcing wire member 30 is nor wrapped in a range from the intermediate position 23 of the inner layer tube 20 to the tip of the tip portion 22. The non-reinforcing portion 25 is composed of a tube portion consisting of the inner layer tube 20 and the outer layer tube 40.

The inner layer tube 20 that constitutes an innermost layer of the catheter tube 10 is formed in a fine and lengthy circular cylindrical shape and provided as an axial center portion of the catheter tube 10. As the material of the inner layer tube 20, various resin materials such as polyamide resin and fluororesin may be used without particular limitation.

The reinforcing wire member 30 wrapped around the inner layer tube 20 is consisted of a single elementary wire (bare wire) composed of a first helical portion 31 helically wound around the inner layer tube 20 from the base portion 21 of the inner layer tube 20 to the intermediate position 23 near the tip portion 22, and a second helical portion 32 helically wound around by being folded in a direction intersecting with the first helical portion 31 at the intermediate position 23, and the first helical portion 31 and the second helical portion 32 are continuously provided as one piece to provide a coil folding structure. A starting end of the reinforcing wire member 30 is adhered to the base portion 21 of the inner layer tube 20 by a first adhesive portion 33. A winding end of the reinforcing wire member 30 is adhered to the base portion 21 of the inner layer tube 20 by a second adhesive portion 34.

The material of the reinforcing wire member 30 is composed of metal or nonmetal. Examples of the shape of the reinforcing wire member 30 include a circular wire or a flat wire. For the metal wire, the wire of various metals such as stainless steel and tungsten steel can be used without any limitation. For the nonmetal wire, the wire of various nonmetals such as fluororesin, aramid fiber, PPS (Polyphenylenesulfide), polyalylate, etc. can be used without any limitation.

The inner layer tube 20 and the outer layer tube 40 provided on the reinforcing wire member 30 are respectively formed in fine and lengthy circular cylindrical shape and constitute the appearance of the catheter tube 10. As the material of the outer layer tube 40, various resin materials such as polyamide resin and fluororesin can be used without particular limitation, similarly to the inner layer tube 20.

The reinforcing wire member 30 may include a single wire which is wrapped around in cross winding, or two or more wires aligned at a predetermined pitch that are wrapped around in cross winding. The outer diameter and winding pitch of the reinforcing wire member 30 are determined according to the type of catheter, the treatment site, and the like. In the illustrated example, the reinforcing wire member 30 is wrapped in such a manner that the adjacent wires are wound with a gap (space), but may be wrapped in such a manner that the adjacent wires are contacting to each other.

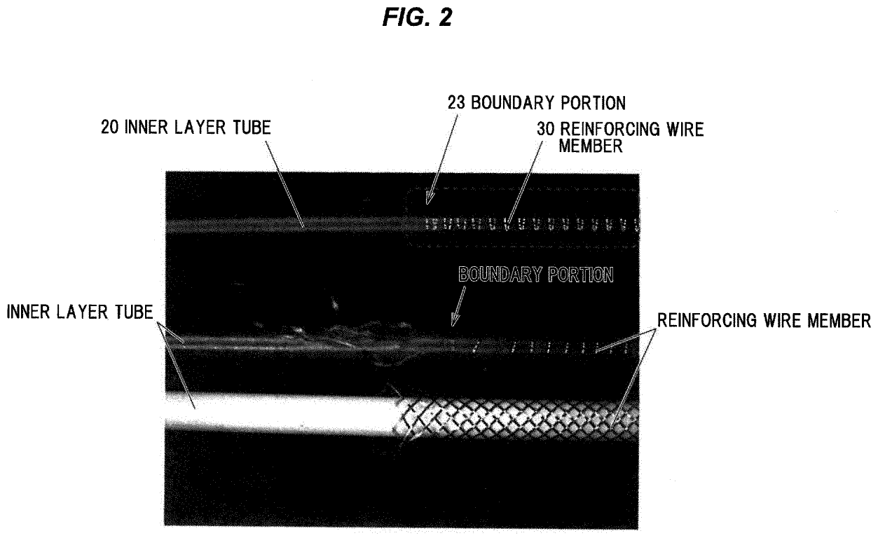

FIG. 2 shows a catheter tube according to the embodiment (located in the upper stage), a catheter tube having a conventional mesh winding reinforcing portion (located at the lower stage), and a catheter tube having a conventional reinforcing portion in which the tip-side is the end of the coil winding (located in the intermediate stage) in comparison.

As shown in FIG. 2, in the case of a conventional reinforcing portion formed by mesh winding, a boundary portion may be opened. Further, at the reinforcing portion where the tip-side is provided as the end of the coil winding, the reinforcing wire member cannot be fixed unless the measures such as welding of the reinforcing wire member are provided.

On the other hand, in the catheter tube according to the present embodiment, the reinforcing wire member 30 is folded at the intermediate position near the tip portion and helically wrapped, so that measures at the boundary portion near the tip portion are unnecessary. That is, exposure and protrusion of the reinforcing wire member 30 to the outside of the catheter tube 10 can be suppressed.

(The First Method for Manufacturing the Catheter Tube 10)

The first method for manufacturing a catheter tube is a method of manufacturing the catheter tube 10 including the inner layer tube 20 as an axial center portion, the reinforcing wire member 30 wrapped around the inner layer tube 20 along an axial outer circumference surface of the inner layer tube 20, and an outer layer tube 40 covering the inner layer tube 20 and the reinforcing wire member 30, and the method comprises setting the inner layer tube 20, helically winding the reinforcing wire member 30 around the inner layer tube 20 from the base portion 21 of the inner layer tube 20 to the intermediate position 23 near the tip portion 22 of the inner layer tube 20 and turning back in a direction intersecting the reinforcing wire member 30 from the intermediate position 23 while rotating the inner layer tube 20 and the reinforcing wire member 30 relatively to each other, and covering the inner layer tube 20 and the reinforcing wire member 30 with the outer layer tube 40.

In the first method for manufacturing the catheter tube 10, the step of helically winding the reinforcing wire member 30 around the inner layer tube 20 while rotating the inner layer tube 20 and the reinforcing wire member 30 relatively to each other, the reinforcing wire member 30 is helically wound around the inner layer tube 20 by rotating the inner layer tube 20. The reinforcing layer to be provided around the inner layer tube 20 is formed by winding the reinforcing wire member 30 around the inner layer tube 20 toward the tip portion 22 and turning back the reinforcing wire member 30 at the tip-side reinforcing end (the intermediate position 23) and winding the reinforcing wire member 30 until the hand-side portion (base portion 21). That is, the reinforcing wire member 30 is wound around the inner layer tube 20 while rotating the inner layer tube 20 and moving the inner layer tube 20 along a longitudinal direction of the inner layer tube 20, and when a travel distance of the inner layer tube 20 along the longitudinal direction reaches the tip-side reinforcing end, the moving direction of the inner layer tube 20 is turned to an opposite direction and the reinforcing wire member 30 is wound around the inner layer tube 20 until the starting position, thereby forming the reinforcing layer.

As a result, a mesh-like reinforcing layer in which there is no exposure or protrusion of the tip-side terminal can be provided at the tip-side reinforcing end, so that an extremely safe reinforcement structure can be obtained that the exposure or protrusion of the elementary wire terminal on the tip-side of the reinforcing wire member 30 can be avoided.

Further, in the so-called microcatheter tube configured to be used in the angiography of peripheral blood vessels, properties such as flexibility and buckling resistance are emphasized over mechanical strength such as destructive pressure resistance and breaking strength. Therefore, the reinforcement structure according to the present embodiment which is composed of a smaller number of elementary wires with a lower pitch is more advantageous than the braided structure.

Furthermore, the catheter tube with a folded and wrapped reinforcing portion has a smaller elongation than the catheter tube with the reinforcing portion of the braided structure. In the braided structure, the elementary wires are braided in such a manner that the positions of the outer side elementary wire and the inner side elementary wire are interchanged at the intersection of the elementary wires, so that the adhesion of the braided portion in the reinforcing layer to the outer layer made of resin in the catheter tube is weaker than the adhesion of helically wound portion in the reinforcing layer to the outer layer made of resin in the catheter tube.

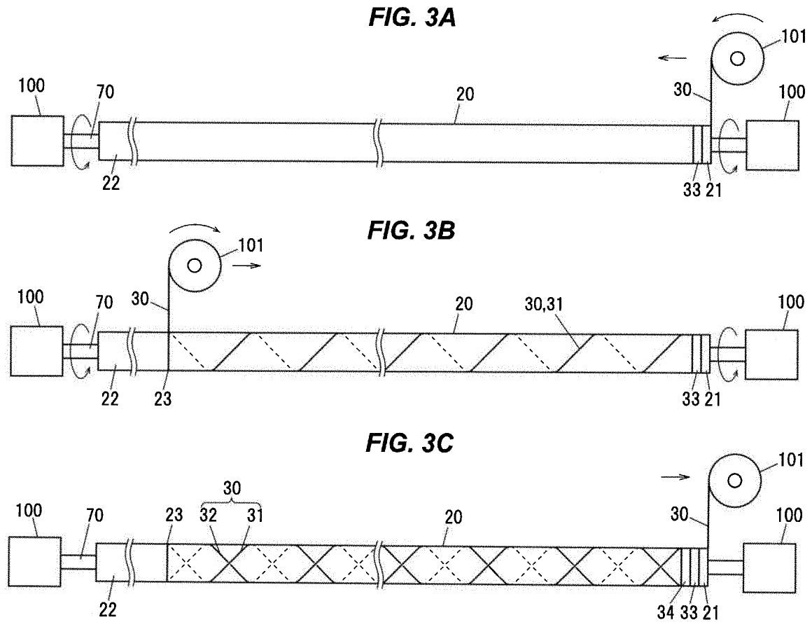

An example of a method for manufacturing a catheter tube 10 will be described with reference to FIGS. 3A to 3C.

In manufacturing the catheter tube 10, a melted resin material for constituting the inner layer tube 20 is formed on an outer circumference of a core wire 70 as extrusion coating with the use of an extrusion molding machine (not shown).

As shown in FIGS. 3A and 3B, both ends of the core wire 70 coated with the inner layer tube 20 are respectively gripped and set in rotatable bobbins 100. The starting end of the reinforcing wire member 30 supplied from a reinforcing wire member supply bobbin 101, which is a rotary advancing/retreating wire member carrier equipped with the reinforcing wire member 30, is adhered to the base portion 21 of the inner layer tube 20 with a first adhesive portion 33.

Next, the inner layer tube 20 is rotated via the core wire 70 by rotating the bobbin 100, and the reinforcing wire member supply bobbin 101 is rotary advanced, so that the reinforcing wire member 30 is helically wound around the inner layer tube 20 from the base portion 21 of the inner layer tube 20 to the intermediate position 23 near the tip portion 22 along the axial center direction outer circumference surface of the inner layer tube 20.

Subsequently, as shown in FIGS. 3B and 3C, the reinforcing wire member supply bobbin 101 is turned to be rotary retreated at the intermediate position 23 of the inner layer tube 20, so that the reinforcing wire member 30 is turned back in a direction intersecting with the direction of the already wound reinforcing wire member 30 and is helically wound around the inner layer tube 20 to the base portion 21 of the inner layer tube 20. After the reinforcing wire member 30 is wrapped around the inner layer tube 20 in cross winding, the reinforcing wire member supply bobbin 101 and the bobbins 100 are stopped. The winding end of the reinforcing wire member 30 is adhered to the base portion 21 of the inner layer tube 20 with a second adhesive portion 34, and the reinforcing wire member 30 on the supply side is cut off. Thereby, the reinforcing wire member 30 is wrapped around in cross winding.

Following the winding step of the reinforcing wire member 30 above, the winding step of the reinforcing wire member 30 may be repeated at a position which is shifted with a predetermined distance toward the tip direction from the intermediate position 23 of the inner layer tube 20.

Next, a melted resin material for constituting the outer layer tube 40 is formed on outer circumferences of the inner layer tube 20 and the reinforcing wire member 30 as extrusion coating with the use of an extrusion molding machine (not shown). Next, the core wire 70 is pulled out and removed.

Next, both ends of the molded body including the inner layer tube 20, the reinforcing wire member 30, and the outer layer tube 40 are cut by a cutting means (not shown) such as a laser cutter. By finishing each of the both ends of the molded body to have a flat surface, the catheter tube 10 is completed.

According to the first manufacturing method, there is no exposure or protrusion of the tip-side terminal of the reinforcing wire member 30 that reinforces the catheter tube 10. Thereby, the molding state of the catheter tube 10 can be kept good, and the catheter tube 10 can be produced with good yield.

(The Second Method for Manufacturing the Catheter Tube 10)

The second method for manufacturing a catheter tube is a method of manufacturing the catheter tube 10 including the inner layer tube 20 as an axial center portion, the reinforcing wire member 30 wrapped around the inner layer tube 20 along an axial outer circumference surface of the inner layer tube 20, and an outer layer tube 40 covering the inner layer tube 20 and the reinforcing wire member 30, and the method comprises setting the inner layer tube 20, helically winding the reinforcing wire member 30 around the inner layer tube 20 from the base portion 21 of the inner layer tube 20 to the intermediate position 23 near the tip portion 22 of the inner layer tube 20 and turning back in a direction intersecting the reinforcing wire member 30 from the intermediate position 23 while rotating the inner layer tube 20 and the reinforcing wire member 30 relatively to each other, and covering the inner layer tube 20 and the reinforcing wire member 30 with the outer layer tube 40.

In the second method for manufacturing the catheter tube 10, the step of helically winding the reinforcing wire member 30 around the inner layer tube 20 while rotating the inner layer tube 20 and the reinforcing wire member 30 relatively to each other, the reinforcing wire member 30 is helically wound around the inner layer tube 20 by rotating the reinforcing wire member 30 around the inner layer tube 20.

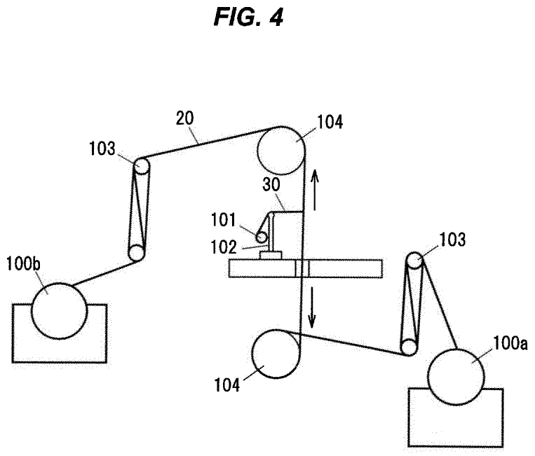

Another example of a method for manufacturing the catheter tube 10 will be described with reference to FIG. 4. In addition, the detailed description is omitted by assigning the same signs to the elements substantially the same as the elements used in the first manufacturing method.

The second manufacturing method is different from the first manufacturing method in that the inner layer tube 20 is moved forward and backward (advanced and retreated) in the axial direction, and the reinforcing wire member 30 supplied from the reinforcing wire member supply bobbin 101 of a wire member carrier 102 is rotated around the axial center of the inner layer tube 20.

The inner layer tube 20 supplied from a supply bobbin 100a via a guide roller 103 is taken up by a take-up capstan 104. The inner layer tube 20 received by the take-up capstan 104 is wound up in the winding bobbin 100b via the guide roller 103. By changing the rotation direction of the take-up capstan 104, the advancing/retreating direction of the inner layer tube 20 can be changed to the up and down directions shown in FIG. 4. As a result, the inner layer tube 20 is moved forward and backward in the axial direction, the reinforcing wire member 30 is rotated around the axial center of the inner layer tube 20, and the reinforcing wire member 30 can be wrapped around the inner layer tube 20 in cross winding.

### Effect of the Embodiments

According to the catheter tube 10 and the method for manufacturing the catheter tube 10 configured as described above, in addition to the above effect, there are the following effects.

(1) Since there is no exposure or protrusion of the tip-side terminal of the reinforcing wire member 30 at the tip portion 22 of the catheter tube 10, safety can be improved and a structure excellent in safety can be manufactured inexpensively.

(2) The reinforcing wire member 30 has a high degree of freedom of winding, so that the catheter tube 10 has excellent flexibility, bendability, rigidity, kink resistance, and the like.

(3) High-strength wire, flat wire, thick wire, etc. can be used, and the reduction in thickness and improved reinforcing structure of the catheter tube 10 can be realized.

(4) Since there is no defect that the tip-side terminal of the reinforcing wire member 30 protrudes from the surface of the catheter tube 10 by the repulsion force of the reinforcing wire member 30 and is exposed therefrom, the yield is stabilized.

(5) A step of removing the tip-side terminal of the reinforcing wire member 30 is unnecessary, so that the production speed and production stability in the winding step of the reinforcing wire member 30 can be improved.

(6) Since the catheter is guided along the guide wire into a complicated blood vessel or introduced into the body from the inside of a guiding catheter, sheath introducer, endoscopic channel, there are problems in that the catheter may be elongated due to sliding resistance on the outer surface and the inner surface, and the operability is hindered, or the like. On the other hand, according to the present embodiment, it is possible to provide the catheter tube 10 excellent in operability with little elongation and the method for manufacturing the same.

Hereinafter, examples and comparative examples will be described as more specific embodiments of the present invention. In addition, a detailed description is omitted by assigning the same signs to the elements used in the above embodiment.

### Example 1

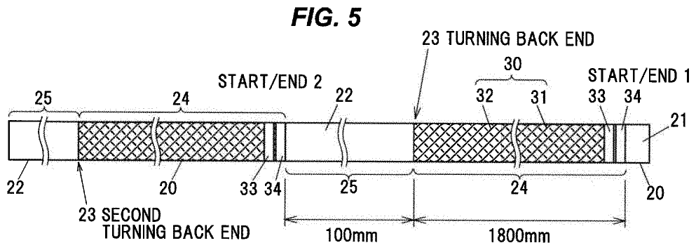

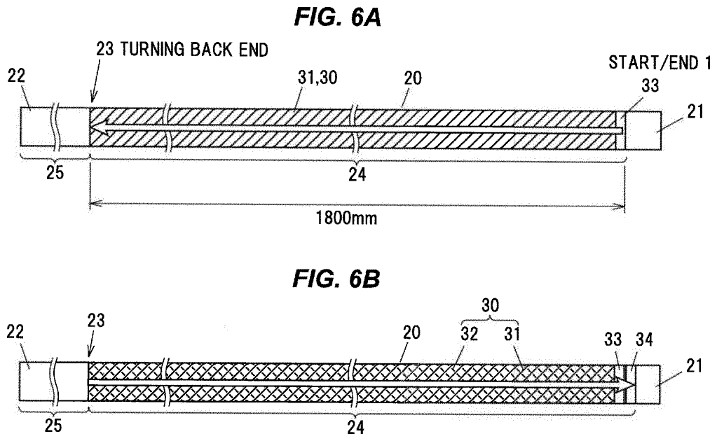

In FIGS. 5, 6A, and 6B, an outer circumference of a silver-plated soft copper core wire having a diameter of 0.43 mm was coated with fluororesin (PTFE) to provide the inner layer tube 20 with a thickness of 0.02 mm, and the inner layer tube 20 was subjected to chemical etching treatment. After fixing two reinforcing wire members 30 made of SUS304WPB and each having a wire diameter of 0.25 mm to the first adhesive portion 33 at an interval of 180 degrees, the reinforcing wire members 30 were wound around the inner layer tube 20 at reinforcing wire member moving speed of 9.6 mm/min and reinforcing wire member winding rate of 80 RPM (winding pitch of 0.12 mm, reinforcing wire member density of 37.30%) for 1800 mm Thereafter, the moving direction of the reinforcing wire members 30 was switched to an opposite direction, and the reinforcing wire members 30 were wound back to the starting position of the winding, and the winding ends of the reinforcing wire members 30 were adhered to the second adhesive portion 34.

By fixing the reinforcing wire members 30 at a position shifted by 100 mm from a folded reinforcing end (i.e., turning back end at the intermediate position 23), and another (next) reinforcing portion 24 was formed. By repeating this process, a continuum in which the reinforcing portions 24 are successively formed on the inner layer tube 20 was produced.

After polyamide elastomer was coated on the inner layer tube 20 to provide the outer layer tube 40 having an outer diameter of 0.64 mm, the continuum was cut in such a manner that a length of the reinforcing portion 24 is 1800 mm and a length of the non-reinforcing portion 25 is 100 mm After stripping both ends by 20 mm, the silver-plated soft copper core wire was pulled out and removed, to thereby provide the catheter tube 10 having an inner diameter of 0.43 mm, and an outer diameter of 0.64 mm.

### Example 2

In FIGS. 5, 6A, and 6B, an outer circumference of a silver-plated soft copper core wire having a diameter of 1.07 mm was coated with 12 nylon resin to provide the inner layer tube 20 with a thickness of 0.07 mm After fixing two reinforcing wire members 30 made of SUS304W1 and each having a wire diameter of 0.03 mm to the first adhesive portion 33 to be aligned in parallel with each other, the reinforcing wire members 30 were wound around the inner layer tube 20 at inner layer tube moving speed of 40 mm/min, reinforcing wire member carrier rotation rate of 80 RPM (winding pitch of 0.5 mm, reinforcing wire member density of 11.70%) for 1800 mm Thereafter, the moving direction of the reinforcing wire members 30 was switched to an opposite direction, and the reinforcing wire members 30 were wound back to the starting position of the winding, and the winding ends of the reinforcing wire members 30 were adhered to the second adhesive portion 34.

By fixing the reinforcing wire members 30 at a position shifted by 100 mm from a folded reinforcing end (the intermediate position 23), and another (next) reinforcing portion 24 was formed. By repeating this process, a continuum in which the reinforcing portions 24 are successively formed on the inner layer tube 20 was produced.

After polyamide elastomer was coated on the inner layer tube 20 to provide the outer layer tube 40 having an outer diameter of 1.43 mm, the continuum was cut in such a manner that a length of the reinforcing portion 24 is 1800 mm and a length of the non-reinforcing portion 25 is 100 mm After stripping both ends by 20 mm, the silver-plated soft copper core wire was pulled out and removed, to thereby provide the catheter tube 10 having an inner diameter of 1.07 mm, and an outer dimeter of 1.43 mm.

### Comparative Example 1

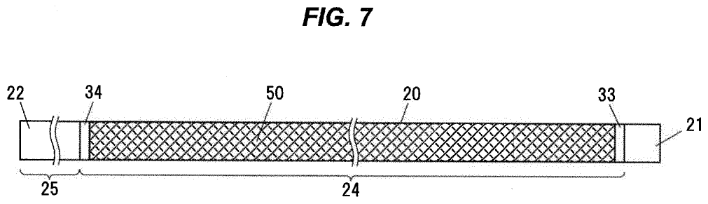

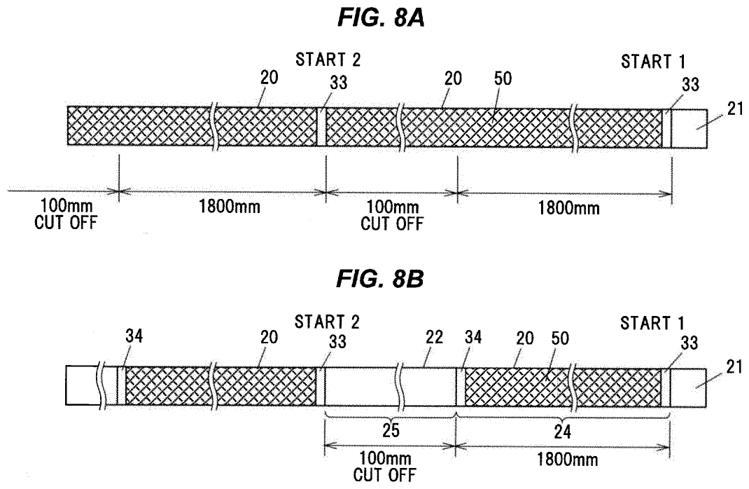

In FIGS. 7, 8A, and 8B, an outer circumference of a soft copper core wire having a diameter of 0.43 mm was coated with fluororesin (PTFB) to provide the inner layer tube 20 with a thickness of 0.02 mm, and the inner layer tube 20 was subjected to chemical etching treatment. Elementary wires of SUS304W1, each having a wire diameter of 0.025 mm, were wrapped around the inner layer tube 20 at inner layer tube moving speed of 65 mm/min, reinforcing wire member carrier rotation rate of 50 RPM (1×16 spindles, winding pitch of 1.3 mm, reinforcing wire member density of 35.50%), to provide a braided portion 50. Thereafter, at intervals of 1900 mm, about 2 mm width portions of the braided portion 50 were adhered to the first and second adhesive portions 33 and 34, and a portion with 100 mm length between the fixed ends of the braided portion 50 was removed with the use of scissors, to provide a non-reinforcing portion 25.

By repeating this process, a continuum in which the reinforcing portions 24 are successively formed on the inner layer tube 20 was produced. After polyamide elastomer was coated on the inner layer tube 20 to provide the outer layer tube 40 having an outer diameter of 0.64 mm, the continuum was cut in such a manner that a length of the reinforcing portion 24 is 1800 mm and a length of the non-reinforcing portion 25 is 100 mm After stripping both ends by 20 mm, the soft copper core wire was pulled out and removed, to thereby provide the catheter tube 10 having an inner diameter of 0.43 mm, and an outer dimeter of 0.64 mm.

### Comparative Example 2

In FIGS. 7, 8A, and 8B, an outer circumference of a soft copper core wire having a diameter of 1.07 mm was coated with 12 nylon resin to provide the inner layer tube 20 with a thickness of 0.07 mm Elementary wires of SUS304W1, each having a wire diameter of 0.03 mm, were wrapped around the inner layer tube 20 at inner layer tube moving speed of 65 mm/min, reinforcing wire member carrier rotation rate of 50 RPM (2×16 spindles, winding pitch of 4.0 mm, reinforcing wire member density of 31.40%), to provide a braided portion 50. Thereafter, at intervals of 1900 mm, about 2 mm width portions of the braided portion 50 were adhered to the first and second adhesive portions 33 and 34, and a portion with 100 mm length between the fixed ends of the braided portion 50 was removed with the use of scissors, to provide a non-reinforcing portion 25.

By repeating this process, a continuum in which the reinforcing portions 24 are successively formed on the inner layer tube 20 was produced. After polyamide elastomer was coated on the inner layer tube 20 to provide the outer layer tube 40 having an outer diameter of 1.43 mm, the continuum was cut in such a manner that a length of the reinforcing portion 24 is 1800 mm and a length of the non-reinforcing portion 25 is 100 mm After stripping both ends by 20 mm, the soft copper core wire was pulled out and removed, to thereby provide the catheter tube 10 having an inner diameter of 1.07 mm, and an outer dimeter of 1.43 mm.

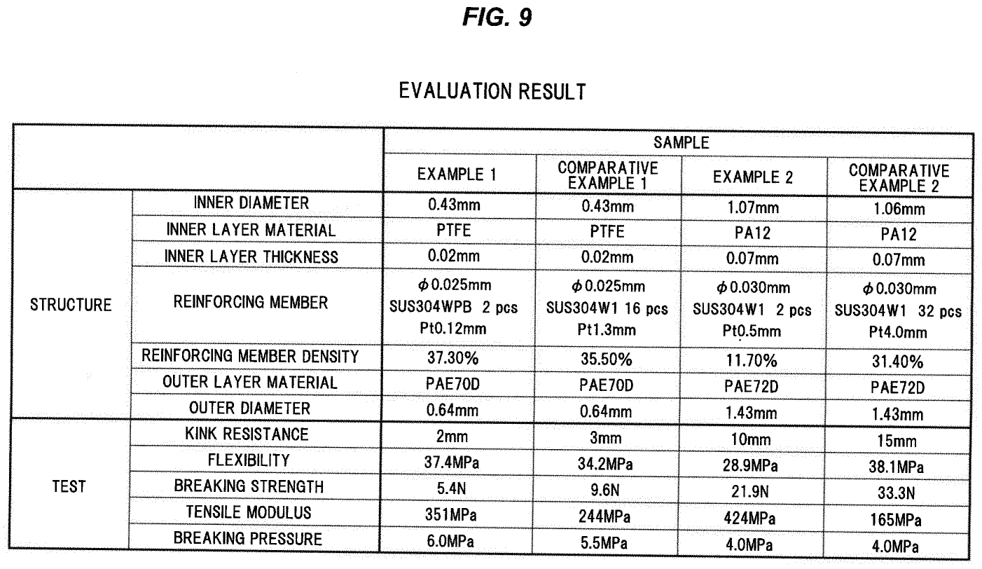

Tests for evaluating kink resistance, flexibility, breaking strength, tensile modulus, and breaking pressure were performed on catheter tubes having coil folding structures in Examples 1 and 2 and catheter tubes having braided (mesh) structures in Comparative Examples 1 and 2. The coil folding structure is a structure equipped with the reinforcing wire member 30 according to the above embodiments. FIG. 9 shows the evaluation results collectively.

(Kink Resistance Test)

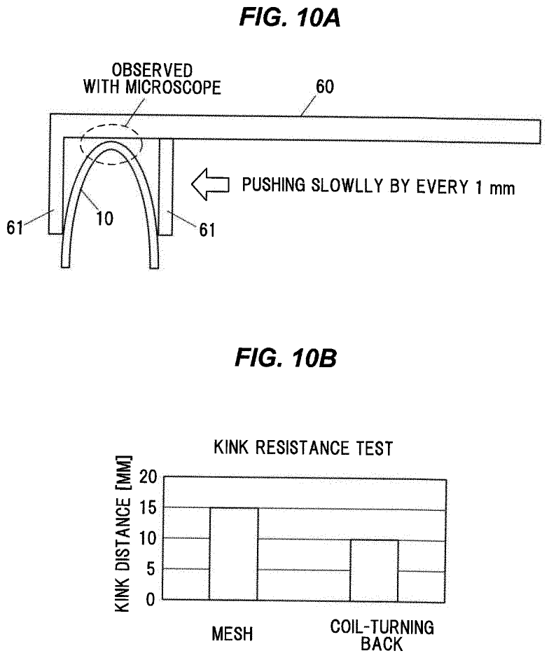

As a method of kink resistance test, as shown in FIG. 10A, the catheter tube 10 was pinched in a U-shape between jaws 61 of a caliper 60. While observing with a microscope, a distance between the jaws 61 of the caliper 60 was shortened 1 mm by 1 mm, and the distance between the jaws 61 of the caliper 60 when completely kinked was measured. FIGS. 9 and 10B show the evaluation results of the difference in crushing between the coil folding structure in Example 2 and the braided structure in Comparative Example 2.

FIG. 10B shows the evaluation results of the kink resistance test of the catheter tube 10 in Example 2 and the catheter tube in Comparative Example 2. The distance between the jaws 61 of the caliper 60 when completely kinked after pressing the caliper 60 is defined as “kink distance”. As clearly understood from FIGS. 9 and 10B, the smaller the measurement value, the higher the kink.

(Flexibility Test)

With the use of a flexibility test apparatus, the distance between the 3D points of the support table was 15 mm for Example 1 and Comparative Example 1 and 50 mm for Example 2 and Comparative Example 2, and the maximum stress at a pushing speed of 100 mm/min was measured. FIG. 9 shows the evaluation results.

(Breaking Strength Test)

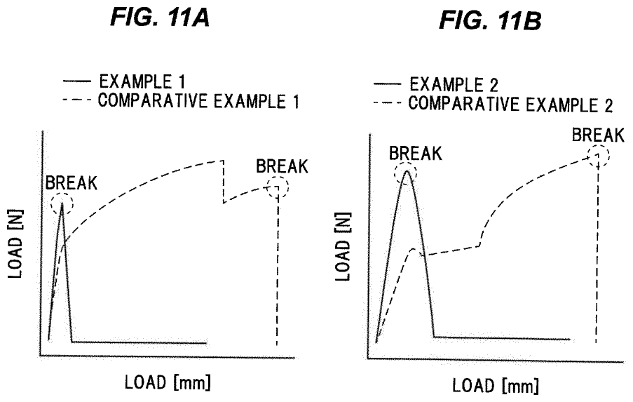

With the use of a universal tensile test apparatus, the test was performed at a tensing speed of 200 mm/min and a chuck distance of 20 mm, and the strength at the time of sample breaking was measured. FIGS. 9, 11A and 11B shows the evaluation results.

(Tensile Modulus Test)

With the use of the universal tensile test apparatus, the tensile modulus (E=ε/σ) was calculated from the stress (ε) when the strain (σ) was applied by 5% by testing at a tensing speed of 200 mm/min and a chuck distance of 20 mm FIG. 9 shows the evaluation results.

(Breaking Pressure Test)

A catheter was connected to a pressure gauged hand-press pump and pressurized at room temperature to measure the pressure that the sample was destroyed. FIG. 9 shows the evaluation results.

As clearly understood from FIGS. 11A and 11B, both the catheter tubes 10 in Examples 1 and 2 have a larger tensile modulus and a smaller elongation than the catheter tubes in Comparative Examples 1 and 2. Therefore, in the operation of the catheter and endoscope, the operability is improved by suppressing the elongation between the base portion 21 on the hand-side (proximal end) and the tip portion 22.

From the above results, the breaking pressure of the catheter tubes in Examples 2 and Comparative Examples 2 is substantially equivalent, but compared to the catheter tubes reinforced with braids in Comparative Examples 1 and 2, the catheter tubes 10 having the coil folding structures as the reinforcing structures in Examples 1 and 2 are excellent in kink resistance and in elongation suppression. Therefore, it can be said that the catheter tube according to the present invention is excellent in safety and also excellent in the characteristics required as a catheter.

As is clear from the above description, representative embodiments, examples, modification, and illustrated examples according to the present invention are illustrated, but the above embodiments, examples, modifications, and illustrated examples do not limit the invention pertaining to the scope of the claim, and can be performed in various embodiments to the extent that the gist is not deviated. Therefore, it should be noted that not all of the combinations of features described in the above embodiments, examples, variants, and illustrated examples are essential for means for solving the problems of invention.

Although the representative embodiments, modifications, and illustrated examples according to the invention have been exemplified, it is obvious from the above description that the invention according to claims is not to be limited to the above-mentioned embodiments, modifications, and illustrated examples and can be implemented in various aspects without departing from the gist of the invention. Therefore, it should be noted that all combinations of the features described in the embodiments, modifications, and illustrated examples are not necessary to solve the problem of the invention.

What is claimed is:

1. A catheter tube, comprising:
an inner layer tube as an axial center portion;
a reinforcing wire member comprising a first helical portion helically wound around the inner layer tube from a base portion of the inner layer tube to an intermediate position near a tip portion, and a second helical portion helically wound around by being folded in a direction intersecting with the first helical portion at the intermediate position; and
an outer layer tube covering the inner layer tube and the reinforcing wire member.

2. The catheter tube according to claim 1, wherein the reinforcing wire member comprises one or two or more wires wound at a predetermined pitch.

3. A method for manufacturing a catheter tube including an inner layer tube as an axial center portion, a reinforcing wire member wrapped around the inner layer tube along an axial outer circumference surface of the inner layer tube, and an outer layer tube covering the inner layer tube and the reinforcing wire member,
the method comprising:
setting the inner layer tube;
helically winding the reinforcing wire member around the inner layer tube from a base portion of the inner layer tube to an intermediate position near the tip portion of the inner layer tube and turning back in a direction intersecting the reinforcing wire member from the intermediate position while rotating the inner layer tube and the reinforcing wire member relatively to each other; and
covering the inner layer tube and the reinforcing wire member with the outer layer tube.

4. The method according to claim 3, further comprising:
repeating the winding of the reinforcing wire member at a position shifted by a predetermined distance in a tip direction from the intermediate position of the inner layer tube after the winding of the reinforcing wire member.
